(19) United States
(12) Patent Application Publication
(10) Pub. No.: US 2010/0160228 A1
(43) Pub. Date: Jun. 24, 2010

(54) METHOD FOR INHIBITING OR STIMULATING ANGIOGENESIS IN AN INDIVIDUAL

(75) Inventors: Johanna Ivaska, Turku (FI); Elina Mattila, Nousiainen (FI)

Correspondence Address: ROTHWELL, FIGG, ERNST & MANBECK, P.C., 1425 K STREET, N.W., SUITE 800, WASHINGTON, DC, 20005, US

(73) Assignee: VALTION TEKNILLINEN TUTKIMUSKESKUS, Espoo (FI)

(21) Appl. No.: 12/601,108

(22) PCT Filed: May 13, 2008
(86) PCT No.: PCT/FI2008/050262

§ 371 Date: Mar. 5, 2010

Related U.S. Application Data
(60) Provisional application No. 60/939,720, filed on May 23, 2007.

(51) Int. Cl. A61K 38/16; A61K 38/08; A61K 31/7088; C07K 7/06; C07K 14/00; A61P 35/00; A61P 9/10

(52) U.S. Cl. 514/12; 514/15; 514/44 R; 530/328; 530/350

(57) ABSTRACT

The invention concerns a method for inhibiting vascular endothelial growth factor receptor 2 (VEGFR2) signalling in an individual by administering an effective amount of an agent capable of activating T cell protein tyrosine phosphatase (TCPTP), and a method for inhibiting angiogenesis and treating or preventing diseases related thereto. Furthermore, this invention concerns a method for stimulating angiogenesis or for treatment or prevention of a disease benefiting from increasing the growth of blood vessels in an individual by inhibiting TCPTP.

### FIELD OF THE INVENTION

This invention relates to a method for inhibiting vascular endothelial growth factor receptor 2 (VEGFR2) signalling in an individual by administering an effective amount of an agent capable of activating T cell protein tyrosine phosphatase (TCPTP), and for inhibiting angiogenesis and treating or preventing diseases related thereto. Furthermore, this invention concerns a method for stimulating angiogenesis or for treatment or prevention of a disease benefiting from increasing the growth of blood vessels in an individual by inhibiting TCPTP.

The invention concerns also a novel peptide and a fusion protein based thereon.

### BACKGROUND OF THE INVENTION

The publications and other materials used herein to illuminate the background of the invention, and in particular, cases to provide additional details respecting the practice, are incorporated by reference.

VEGF (vascular endothelial growth factor) is a major growth factor for endothelial cells. It regulates endothelial cell function in the formation of new blood vessels during development as well as in adults (e.g. during wound healing and in proliferative diseases like cancer) (1). In the vascular endothelium VEGF binds to two high-affinity tyrosine kinase receptors VEGFR1 and VEGFR2. VEGFR2 appears to be the main mediator of VEGF-induced endothelial proliferation, survival, migration, tubular morphogenesis, and sprouting in adults (2). The effects of VEGF are mainly controlled by its availability and by the activity of VEGFR signalling. Upon binding of VEGF the receptors dimerize, become autophosphorylated, and recruit signal transduction molecules such as PLC, PI3K and Ras (3). On the other hand, negative regulation of VEGFR2 signalling is equally important in limiting the response of VEGF in target cells. In fact, inhibition of neovascularization is a promising new way of treating diseases such as cancer, certain retinopathies and other angiogenetic diseases.

During the different steps of angiogenesis, the endothelial cell encounters dramatically different extracellular matrices, ranging from the mature basement membrane below the contact-inhibited endothelial cells to the type I collagen-rich stroma around the sprouting sparse cells. Integrin-mediated adhesion to the extracellular matrix (ECM) provides permissive signals to cells by supporting migration, cell survival, and signalling by receptor tyrosine kinases (RTKs) (4). Different integrins have the capacity to form complexes with growth factor receptors on the cell membrane and regulate their activation and internalization. For example, integrin-mediated adhesion alone can activate growth factor receptor phosphorylation in the absence of ligands (5) and integrins bind directly to growth factor receptors like EGFR and cMET (5,6). Adhesion of the endothelial cells to extracellular matrix molecules also profoundly affects their angiogenetic properties. For instance, binding of endothelial cells to vitronectin by αvβ3 integrin positively regulates VEGFR2 activation via direct interaction between the two molecules. It has also been known for a long time that collagenous matrix can inhibit endothelial growth (7). However, the molecular nature of the matrix-dependent integrin-mediated signals that inhibit angiogenesis is not understood.

We recently reported that a collagen-binding integrin α1β1 negatively regulates EGFR (epidermal growth factor receptor) phosphorylation in malignant epithelial cells. This was the first demonstration of an inhibitory role for ECM in signalling by RTKs. The α1 integrin effect was mediated via coupling to TCPTP (T-cell protein tyrosine phosphatase) (8). TCPTP is a 45 kDa protein expressed, despite of its name, in several cell types.

### SUMMARY OF THE INVENTION

The present invention is based on a novel study where we surprisingly found that TCPTP is present in endothelial cells and that a ligation of a collagen-binding integrin or TCPTP activation could restrain VEGFR2 signalling and inhibit biological responses to VEGF. We found that TCPTP dephosphorylates and silences VEGFR2 and devised a peptide from the cytoplasmic domain of integrin α1 to be used as a tool to inhibit VEGFR2 signalling via the activation of TCPTP. The data obtained in our study indicate that endothelial cell growth during different steps of angiogenesis is regulated by microenvironmentally controlled phosphatase activities.

Thus, according to one aspect, this invention concerns the use of an agent capable of activating T cell protein tyrosine phosphatase (TCPTP) in the manufacture of a pharmaceutical composition for use in a method for inhibiting vascular endothelial growth factor receptor 2 (VEGFR2) signalling in an individual.

In another aspect, the invention concerns the use of an agent capable of activating T cell protein tyrosine phosphatase (TCPTP) in the manufacture of a pharmaceutical composition for use in a method for inhibiting angiogenesis or for treatment or prevention of a disease benefiting from suppression of the growth of blood vessels in an individual.

According to a third aspect, the invention concerns the use of an agent capable of inhibiting T cell protein tyrosine phosphatase (TCPTP) in the manufacture of a pharmaceutical composition for use in a method for stimulating angiogenesis or for treatment or prevention of a disease benefiting from increasing the growth of blood vessels in an individual.

According to a fourth aspect, the invention concerns a peptide comprising the amino acid sequence RPLKKKMEK (SEQ ID NO 1), wherein all the amino acids are D-amino acids, or a vector being capable of expressing said peptide in a mammalian cell.

In a fifth aspect, this invention concerns a method for inhibiting vascular endothelial growth factor receptor 2 (VEGFR2) signalling in an individual, by administering to said individual an effective amount of an agent capable of activating T cell protein tyrosine phosphatase (TCPTP).

In a sixth aspect, the invention concerns a method for inhibiting angiogenesis or for treatment or prevention of a disease benefiting from suppression of the growth of blood vessels in an individual by administering to said individual an effective amount of an agent capable of activating T cell protein tyrosine phosphatase (TCPTP).

In a seventh aspect, the invention concerns a method for stimulating angiogenesis or for treatment or prevention of a disease benefiting from increasing the growth of blood vessels in an individual by administering to said individual an effective amount of an agent capable of inhibiting T cell protein tyrosine phosphatase (TCPTP).

In an eight aspect, this invention concerns an agent capable of activating T cell protein tyrosine phosphatase (TCPTP) for use in a method for inhibiting vascular endothelial growth factor receptor 2 (VEGFR2) signalling in an individual.

In a ninth aspect, the invention concerns an agent capable of activating T cell protein tyrosine phosphatase (TCPTP) for use in a method for inhibiting angiogenesis or for treatment or prevention of a disease benefiting from suppression of the growth of blood vessels in an individual.

In a tenth aspect, the invention concerns an agent capable of inhibiting T cell protein tyrosine phosphatase (TCPTP) for use in a method for stimulating angiogenesis or for treatment or prevention of a disease benefiting from increasing the growth of blood vessels in an individual.

### BRIEF DESCRIPTION OF THE DRAWINGS

FIG. 1. Adhesion of HUVEC to collagen activates TCPTP. (A) Immunoblotting of α1 and β1 integrins, TCPTP and VEGFRs from HUVEC lysates (HeLa lysates and tubulin blots are shown as controls) (B) Phosphatase activities (mean±SD; n=3) in control (IgG), TCPTP, and SHP2 immunoprecipitates from HUVEC grown on collagen and gelatin. The immunoprecipitates were also resolved on SDS-PAGE gels and probed for SHP2 and TCPTP. A representative gel out of three with similar results is shown. *, p<0.05; ***, p<0.005

FIG. 2. TCPTP binds to VEGFR2, but not to VEGFR1, and dephosphorylates it in a phospho-site specific manner. (A) Microscopy images from two-color immunofluorescence stainings show co-localization of integrin α1β1 with TCPTP and VEGFR2 (arrows) in HUVEC adhering to collagen. Bar, 10 μm. (B) HEK293 cells transiently transfected with VEGFR2 and substrate-trapping mutant of TCPTP (TCPTP-D182A) were treated with VEGF (100 ng/ml, 15 min) and immunoprecipitated (IP) with anti-TCPTP or control (IgG) antibodies and blotted as indicated. Aliquots of the lysates were also analysed to control for equal protein expression. (C) HEK293 cells were transfected with VEGFR2, treated with VEGF and immunoprecipitated (IP) with anti-VEGFR2. Immunoprecipitates were incubated in the presence of recombinant TCPTP or buffer control, and immunoblotted using phospho-specific VEGFR2 antibodies. Total VEGFR2 was blotted as a control. Representative experiments out of 3 with similar results are shown.

FIG. 3. TCPTP activity controls VEGF-dependent responses in endothelial cells. (A) HUVEC were transfected with the constitutively active TCPTP (TC37) or vector control (pCG), stimulated or not with VEGF, and stained for VEGFR2 (red) and nuclei (blue) after fixation and permeabilization. Arrows indicate VEGFR2 vesicles. (B) HUVEC were treated as in (A) and subjected to trypsin digestion on ice. VEGFR2 protected from trypsin (=internalized pool) was detected by immunoblotting. The intensities of VEGFR2 bands were quantified and are expressed relative to pCG-VEGF control. A representative experiment out of 2 with similar results is shown. (C) HUVEC were transfected with no oligo, siRNA against TCPTP or a control siRNA, and TCPTP expression was measured using immunoblotting. Cells were treated with 80 ng/ml VEGF and proliferation of sparse cells plated on gelatin was assayed after 16 h using BrdU-based assay (mean±SD; n=4-6; *, p<0.05). (D) HUVEC were transfected as in (A) and chemotaxis towards VEGF was measured using a Transwell assay for 4 hours. Cells adhering to the bottom of the filter were fixed and stained with crystal violet. Number of migrated cells was counted (mean±SEM, n=3).

FIG. 4. α1 integrin activates TCPTP and attenuates VEGFR2 signalling. (A) Serum-starved subconfluent HUVEC were treated for 1 h with α1 integrin cytoplasmic tail fusion peptide (α1TAT) or TAT-scramble control fusion peptide (ScrTAT) (both at 200 nM) followed by VEGF-induction (100 ng/ml, 15 min) where indicated. Cell lysates were resolved on SDS-PAGE and immunoblotted for phosphorylated VEGFR2 (or tubulin as a control). (B) HUVEC were treated as in (A) and VEGFR2 kinase activity (mean±SD, n=3) was measured from the cell lysates. **, p<0.01. (C) Recombinant, purified TCPTP was incubated with α1 or α2 cytoplasmic tail peptides (L-amino acids) or with α1 and scrambled TAT peptides (D-amino acids) and analyzed for phosphatase activity (mean±SD, n=3).

FIG. 5. Controlled TCPTP activation inhibits VEGF-driven capillary formation, chemokinesis and chemotaxis. (A) HUVEC were cultured in fibrin gels and treated with or without VEGF and TAT peptides (200 nM). After 24 h the formation of capillary tubes was semiquantitatively scored. (B) HUVEC were plated on gelatin in the presence of the peptides (200 nM) or left untreated for 1 h. The cells were stimulated as indicated and migration of individual cells was tracked using time-lapse microscopy. Cumulative migration distance was plotted for randomly picked individual cells. (C) HUVEC were treated with 200 nM peptides and the transmigration assay was performed as in FIG. 3D. (D) HUVEC were transfected with siRNA against TCPTP or a control siRNA, and TCPTP expression was analysed with immunofluorescence. Equal exposures of TCPTP (green) and DAPI (blue) stainings are shown. 48 h post-transfection cells were subjected to transmigration assay as in (D).

FIG. 6. PAE cells stably expressing VEGFR1 were transfected with the substrate-trapping mutant of TCPTP (TCPTP-D182A), treated with VEGF (100 ng/ml, 15 min) or left untreated and immunoprecipitated (IP) with anti-TCPTP or control (IgG) antibodies and blotted as indicated. Aliquots of the lysates were also analysed to control for equal protein expression.

FIG. 7. TCPTP activation through the cytoplasmic domain of α1 integrin inhibits internalization of VEGFR2. (A) Serum-starved subconfluent HUVEC were treated for 1 h with α1 integrin cytoplasmic tail fusion peptide (α1TAT) or TAT-scramble control fusion peptide (ScrTAT) (both at 200 nM) followed by VEGF-induction (100 ng/ml, 15 min) where indicated. The cells were then stained for VEGFR2 (red) and nuclei (blue). VEGFR2 staining in the perinuclear area of cells (mean±SEM, n=30) was analyzed from blinded samples using densitometry and scored from 0 to 4 (0=no staining, 4=highest expression). (B) HUVEC were treated as in (A) and subjected to trypsin digestion on ice. VEGFR2 protected from trypsin (=internalized pool) was detected by immunoblotting. The intensity of VEGFR2 bands was quantified using densitometry and expressed as arbitrary units. A representative experiment out of 3 with similar results is shown.

FIG. 8. VEGF-induced capillary formation is attenuated by α1-integrin. HUVEC were plated on gelatin, overlayed by collagen and treated with or without VEGF and TAT peptides (200 nM). After 24 h the number of closed polygons formed by endothelial tubes was enumerated microscopically. The mean scores from a representative assay are shown.

FIG. 9. TCPTP activation by α1-peptide inhibits VEGF induced chemokinesis. HUVEC were plated on gelatin in the presence of the peptides (200 nM) or left untreated for 1 h. The cells were stimulated as indicated and migration of individual cells was tracked using time-lapse microscopy. Representative images of individual cells overlayed with their migration patterns (different colours) at the indicated time points are shown.

FIG. 10. Activation of TCPTP does not inhibit VEGF-responses in confluent HUVEC. (A) Serum-starved HUVEC were plated to confluency on gelatin and treated for 1 h with α1 integrin cytoplasmic tail fusion peptide (α1TAT) or TAT-scramble control fusion peptide (ScrTAT) (both at 200 nM) or not treated, followed by VEGF-induction (15 min) when indicated. Cell lysates were immunoblotted for phosphorylated VEGFR2 (or tubulin as a control). (B) Confluent HUVEC on gelatin were treated with VEGF in the presence or absence of α1TAT or ScrTAT peptide and the number of live cells was detected using WST-1 reagent (mean±SD, n=3). (C) α1TAT treatment has no effect on internalization of VEGFR2 in confluent HUVEC. HUVEC were plated to confluency on gelatin, stimulated or not with the TAT peptides (200 nM) and VEGF, and treated with trypsin on ice. Cells were lysed after inactivation of trypsin and the lysates were immunoblotted for detection of VEGFR2 protected from trypsin (=internalized pool). The intensity of VEGFR2 bands was quantitated using densitometry (a representative experiment out of 3 with similar results is shown).

FIG. 11 shows the mRNA sequence (SEQ ID NO 2) of TCPTP where the binding sites for two siRNA:s are marked.

### DETAILED DESCRIPTION OF THE INVENTION

### Definitions

The term “treatment” or “treating” shall be understood to include complete curing of a disease or disorder, as well as amelioration or alleviation of said disease or disorder.

The term “prevention” shall be understood to include complete prevention, prophylaxis, as well as lowering the individual's risk of falling ill with said disease or disorder.

The term “individual” refers to a human or animal subject.

The term “effective amount” is meant to include any amount of an agent according to the present invention that is sufficient to bring about a desired therapeutical result, especially upon administration to an animal or human subject.

The term “inhibiting” or “inhibition” shall be understood to include not only complete inhibition but also any grade of suppression.

The term “peptide” shall be understood to include peptides of L-amino acids, D-amino acids or both unless the form of the amino acids is explicitly expressed.

### Preferable Embodiments

The agent to be used for activating of TCPTP in order to inhibit VEGRF2 signalling can either be a small molecule able to activate TCPTP, or a peptide comprising the amino acid sequence RPLKKKMEK (SEQ ID NO 1). This sequence is specific for the cytoplasmic tail of alpha-1-integrin. The peptide can, according to one embodiment, be exactly the amino acid sequence RPLKKKMEK (SEQ ID NO 1), which could be administered to the cell by microinjection, for example.

Alternatively, the peptide can be a longer chain encompassing the amino acid sequence RPLKKKMEK (SEQ ID NO 1). According to a particularly preferable embodiment, the peptide (the chain RPLKKKMEK (SEQ ID NO 1) or a longer chain encompassing the same) can be part of a cell membrane permeable fusion protein. As an example of such a fusion protein can be mentioned a fusion protein comprising a protein transduction domain of HIV transcriptional transactivator (TAT) protein and the amino acid sequence RPLKKKMEK (SEQ ID NO 1). As a specific example of such fusion proteins can be mentioned YGRKKRRQRRRWKLGFFKRPLKKKMEK (SEQ ID NO 3). The sequence YGRKKRRQRRR (SEQ ID NO 4) is derived from TAT and the sequence WKLGFFK (SEQ ID NO 5) is derived from the integrin (typical for any alpha-integrin). As another example of a suitable cell membrane permeable fusion protein can be mentioned a fusion protein of the antennapedia peptide RQIKIWFQNRRMKWKK (SEQ ID NO 6) and the amino acid sequence RPLKKKMEK (SEQ ID NO 1). The sequence of such a fusion protein is RQIKIWFQNRRMKWKKWKLGFFKRPLKKKMEK (SEQ ID NO 7).

According to a particularly preferred embodiment, all the amino acids in the RPLKKKMEK (SEQ ID NO 1) are D-amino acids. In case the peptide RPLKKKMEK (SEQ ID NO 1) is a part of a fusion protein, preferably all amino acids of such a fusion protein are D-amino acids.

According to a further alternative, the agent to be administered can be a vector, comprising a nucleic acid being capable of expressing the desired peptide in a mammalian cell. The nucleic acid can be inserted in a DNA sequence, an RNA sequence or in a viral vector. Such a viral vector is typically based on an adenovirus, an alphavirus, adeno-associated virus, a retrovirus or a herpes virus. For expression of peptides in cells, see for example M Parsons et al., Molecular and Cellular Biology, August 2002, p. 5897-5911.

As non-restricting examples of suitable small molecules for activating TCPTP can be mentioned Ruthenium Red, Spermidine, Mitoxantrone and MDL-26,630 trihydrochloride.

Inhibiting VEGFR2 signalling by administering an effective amount of an agent capable of activating TCPTP is useful for inhibiting angiogenesis or for treatment or prevention of a disease benefiting from suppression of the growth of blood vessels in an individual. As non-restricting examples of diseases that could be treated or prevented by this method can be mentioned retinopathy, age related macular degeneration, hemangioma, arthritis, psoriasis, atherosclerosis and cancer. This method is very effective in destroying the vasculature of tumors.

The peptide comprising the sequence RPLKKKMEK (SEQ ID NO 1) is preferably brought in a form which allows permeation through the cell membrane. Such a peptide can be admixed with any carrier that is suitable for parenteral administration of the composition. For example, the peptide can be complexed with a lipid, packed in a liposome, incorporated in a cyclodextrin or other complexing agent, a bioresorbable polymer or other suitable carrier for controlled release administration, or encompassed in a nanoparticle or hydrogel.

Alternatively, an expression vector encompassing a nucleic acid encoding a peptide comprising the amino acid sequence RPLKKKMEK (SEQ ID NO 1) can be administered to the individual. The nucleic acid can encode the exact sequence RPLKKKMEK (SEQ ID NO 1) or a longer peptide comprising this sequence. The vector can, for example, be complexed with a lipid, packed in a liposome, incorporated in a cyclodextrin or other complexing agent, a bioresorbable polymer or other suitable carrier for controlled release administration, or encompassed in a nanoparticle or hydrogel.

The therapeutically effective amount of the peptide or expression vector to be given to a patient in need of such treatment may depend upon a number of factors including, for example, the age and weight of the patient, the precise condition requiring treatment and its severity, and the route of administration. The precise amount will ultimately be at the discretion of the attending physician. Thus, practice of the present invention may involve any dose, combination with other therapeutically effective drugs, pharmaceutical formulation or delivery system for parenteral administration. The peptide or expression vector can be administered systemically or locally. As suitable routes of administration can be mentioned intravenous, intramuscular, subcutaneous injection, inhalation, topical, ocular, sublingual, nasal, rectal, intraperitoneal delivery and iontophoresis or other transdermal delivery systems.

According to a further preferable embodiment, VEGFR2 signalling, and consequently angiogenesis, can be stimulated in an individual by administering an effective amount of an agent capable of inhibiting TCPTP. This method is useful for treatment or prevention of a disease benefiting from increasing the growth of blood vessels in an individual. As non-restricting examples of diseases that could be treated or prevented by this method can be mentioned coronary artery disease, peripheral arterial occlusion or another disease benefiting from promoting of neovascularisation.

As examples of agents for inhibiting TCPTP can be mentioned i) a small inhibitory RNA (siRNA) oligonucleotide capable of inhibiting TCPTP expression ii) a vector being capable of expressing said inhibitory RNA as a hairpin RNA (shRNA) in mammalian cell, or iii) a small molecule able of inhibiting TCPTP.

The application of siRNA:s has become important in the development of new therapies in the last years. O Heidenreich presents an overview of pharmaceutical applications in the article “Forging therapeutics from small interfering RNAs in European Pharmaceutical Review Issue 1, 2005. The principle has particularly been suggested for the treatment of tumors and carcinomas, sarcomas, hypercholesterolemia, neuroblastoma and herpetic stromal keratitis.

The principle of siRNA is extensively presented in literature. As examples can be mentioned the US patent publications 2003/0143732, 2003/0148507, 2003/0175950, 2003/0190635, 2004/0019001, 2005/0008617 and 2005/0043266. An siRNA duplex molecule comprises an antisense region and a sense strand wherein said antisense strand comprises sequence complementary to a target region in an mRNA sequence encoding a certain protein, and the sense strand comprises sequence complementary to the said antisense strand. Thus, the siRNA duplex molecule is assembled from two nucleic acid fragments wherein one fragment comprises the antisense strand and the second fragment comprises the sense strand of said siRNA molecule. The sense strand and antisense strand can be covalently connected via a linker molecule, which can be a polynucleotide linker or a non-nucleotide linker. The length of the antisense and sense strands are typically about 19 to 21 nucleotides each. Typically, the antisense strand and the sense strand both comprise a 3′-terminal overhang of a few, typically 2 nucleotides. The 5′-terminal of the antisense is typically a phosphate group (P). The siRNA duplexes having terminal phosphate groups (P) are easier to administrate into the cell than a single stranded antisense. In the cell, an active siRNA antisense strand is formed and it recognizes a target region of the target mRNA. This in turn leads to cleaving of the target RNA by the RISC endonuclease complex (RISC=RNA-induced silencing complex) and also in the synthesis of additional RNA by RNA dependent RNA polymerase (RdRP), which can activate DICER and result in additional siRNA duplex molecules, thereby amplifying the response.

The term “complementary” means that the nucleotide sequence can form hydrogen bonds with the target RNA sequence by Watson-Crick or other base-pair interactions. The term shall be understood to cover also sequences which are not 100% complementary. It is believed that also lower complementarity might work. However, 100% complementarity is preferred.

The siRNA shall, when used as a pharmaceutical, be introduced in a target cell. The delivery can be accomplished in two principally different ways: 1) exogenous delivery of the oligonucleotide or 2) endogenous transcription of a DNA sequence encoding the oligonucleotide, where the DNA sequence is located in a vector.

Normal, unmodified RNA has low stability under physiological conditions because of its degradation by ribonuclease enzymes present in the living cell. If the oligonucleotide shall be administered exogenously, it is highly desirable to modify the molecule according to known methods so as to enhance its stability against chemical and enzymatic degradation.

Modifications of nucleotides to be administered exogenously in vivo are extensively described in the art. Principally, any part of the nucleotide, i.e. the ribose sugar, the base and/or internucleotidic phosphodiester strands can be modified. For example, removal of the 2′-OH group from the ribose unit to give 2′-deoxyribosenucleotides results in improved stability. Prior discloses also other modifications at this group: the replacement of the ribose 2′-OH group with alkyl, alkenyl, allyl, alkoxyalkyl, halo, amino, azido or sulfhydryl groups. Also other modifications at the ribose unit can be performed: locked nucleid acids (LNA) containing methylene linkages between the 2′- and 4′-positions of the ribose can be employed to create higher intrinsic stability. Furthermore, the internucleotidic phosphodiester linkage can, for example, be modified so that one or more oxygen is replaced by sulfur, amino, alkyl or alkoxy groups. Also the base in the nucleotides can be modified. Preferably, the oligonucleotide comprises modifications of one or more 2′-hydroxyl groups at ribose sugars, and/or modifications in one or more internucleotidic phosphodiester linkages, and/or one or more locked nucleic acid (LNA) modification between the 2′- and 4′-position of the ribose sugars. Particularly preferable modifications are, for example, replacement of one or more of the 2′-OH groups by 2′-deoxy, 2′-O-methyl, 2′-halo, e.g. fluoro or 2′-methoxyethyl. Especially preferred are oligonucleotides where some of the internucleotide phoshodiester linkages also are modified, e.g. replaced by phosphorothioate linkages.

It should be stressed that the modifications mentioned above are only non-limiting examples.

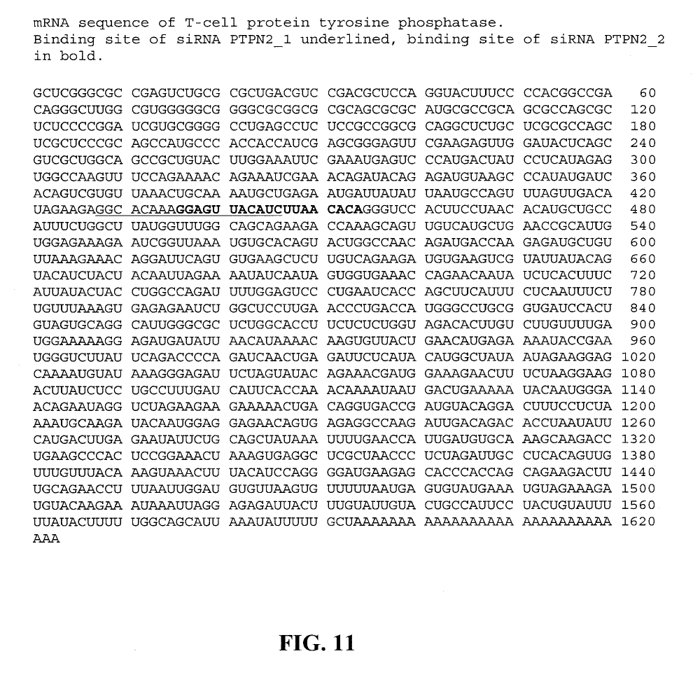

Examples of useful siRNA:s and vectors are disclosed in reference 8, paragraph Transfections. Two different annealed siRNAs targeting TCPTP (ggcacaaaggaguuacauctt and ggaguuacaucuuaacacatt) were disclosed. These sequences (except for the overhangs tt) represent the sense strands of the duplexes, which also comprise antisense strands which complementary to the sense strands. The target sites for these siRNA:s are indicated as underlined or bold, respectively, in FIG. 11.

However, also other useful target regions at the target RNA can be used. A useful target region can easily be identified by using any of the numerous academic or commercially affiliated algorithms that have been developed to assist scientists to locate utilizable siRNA sequences. As examples of such software systems can be mentioned siDirect (http://design.RNAi.jp/) (Nucleic Acids Res. 2004 Jul. 1; 32: W124-9); TROD (T7 RNAi Oligo Designer (http://www.cellbio.unige.ch/RNAi.html; Nucleic Acids Res. 2004 Jul. 1; 32: W121-3); DEQOR (http://cluster-1.mpi-cbg.de/Deqor/deqor.html; Nucleic Acids Res. 2004 Jul. 1; 32: W113-20) or programs available at http://www.genscript.com; http://www.genscript.com/rnai.html#design or http://www.genscript.com/sirna_ca.html#design; Bioinformatics 2004 Jul. 22; 20(11)1818-20. An essential criterion of the tools is to achieve siRNA:s with maximum target-specificity for mammalian RNA interference where off-target gene silencing is avoided. The usefulness of any sequence identified by such algorithms should thereafter be verified by experiments.

TCPTP shRNA construct was made by cloning the sequence corresponding to the functional TCPTP1 oligo into pRNA-U6.1/Neo (Genscript, Piscataway, N.J.) according to the manufacturer's instructions.

The invention will be illuminated by the following non-restrictive Experimental Section.

### Experimental Section

In the study disclosed below we show that T-cell protein tyrosine phosphatase (TCPTP) is expressed in human endothelial cells and that it interacts with VEGFR2. TCPTP dephosphorylates VEGFR2 in a phosphosite-specific manner, blocks its kinase activity and prevents its internalization from the surface of endothelial cells. siRNA-mediated down-regulation of TCPTP results in augmented proliferation of endothelial cells upon VEGF stimulation, whereas constitutively active TCPTP inhibits biological responses to VEGF. TCPTP activity is induced upon integrin-mediated binding of endothelial cells to collagen matrix. TCPTP can also be activated in endothelial cells using cell-permeable peptides from the cytoplasmic tail of the collagen-binding integrin α1. This controlled activation of TCPTP results in inhibition of VEGF-triggered endothelial cell proliferation, chemokinesis, chemotaxis, and capillary formation. Importantly TCPTP-dependent inhibition of VEGF signalling specifically targets actively growing endothelial cells. These data provide novel mechanistic insights into the regulation of VEGF signalling by showing that the growth, migration, and differentiation of human endothelial cells are controlled through matrix-dependent TCPTP phosphatase activity. Moreover, they imply that controlled activation of TCPTP is a potential new way for regulating angiogenesis in different diseases.

### Materials and Methods

Antibodies, plasmids and reagents. Antibodies against TCPTP (mAb CF4, Calbiochem and polyclonal Ab, BD Pharmingen), VEGFR1 (Santa Cruz), total VEGFR2 (55B11), and phosphotyrosines 996, 1175 and 1214 of VEGFR2 (all from Cell Signaling Technology), α1-integrin (mAb 1973 (clone FB12) and Ab1934, Chemicon), β1-integrin (mAb 2252, Chemicon), tubulin (Hybridoma Bank), and control mouse IgG were used. HRP- and Alexa conjugated second-stage reagents were used, as appropriate. A plasmid containing a 37 kDa constitutively active form of TCPTP (TC37 in pcDNA3.1(+)), a substrate-trapping mutant of TCPTP (pCG-TC45-D182A), the control pCG-vector and VEGFR2 expression plasmid have been described (24,25).

siRNA against TCPTP and a control siRNA (8) were from Ambion. Peptides containing the cytoplasmic tails of α1 or α2 integrins, the α1-tail, the sequence of which is RPLKKKMEK (SEQ ID NO 1), fused to the 11 amino acid long TAT peptide (the sequence of which is YGRKKRRQRRR, (SEQ ID NO 4)) and scrambled TAT peptides (8) were synthesized by Innovagen. α1TAT and ScrTAT peptides were also synthesized with D-amino acids. The sequences of the α1TAT and ScrTAT peptides were as follows: α1TAT:

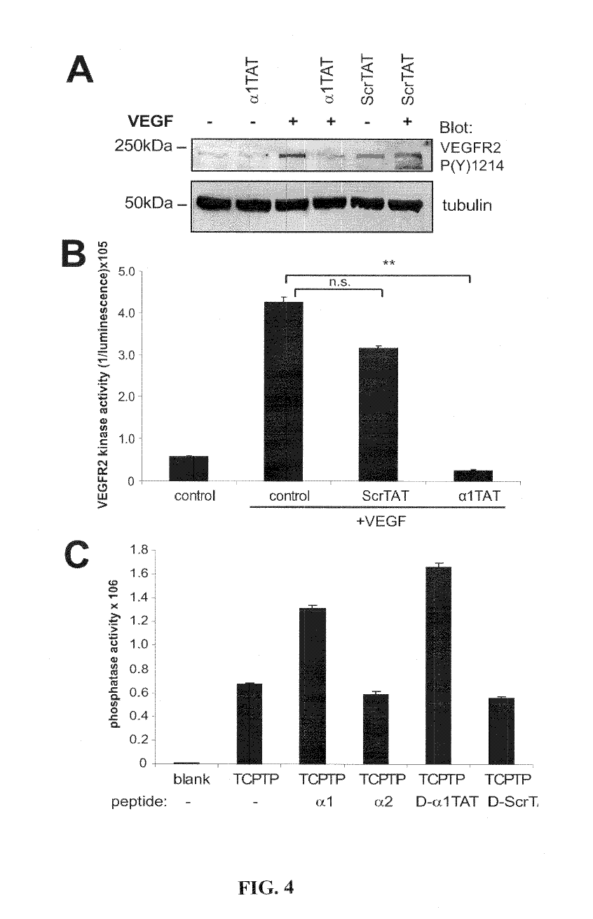

YGRKKRRQRRRWKLGFFKRPLKKKMEK (SEQ ID NO 3) (where YGRKKRRQRRR (SEQ ID NO 4) is the 11 aa long TAT peptide, RPLKKKMEK (SEQ ID NO 1) is the α1-tail of the integrin and WKLGFFK (SEQ ID NO 5) is derived from the integrin); and
Scr-TAT: YGRKKRRQRRRLKGWRFKLKPKFKEMK (SEQ ID NO 8). In the peptides used in the experiments, all the amino acids were D-amino acids except for the peptides α1 and α2 shown as the third and fourth columns (from left) in FIG. 4C. Recombinant TCPTP was produced and purified as described (8). Human recombinant VEGF 165 was purchased from Peprotech.

Cells. HEK293 and HeLa cells were from ATCC and maintained in DMEM, 10% FBS, 2 mM L-glutamine, 5% CO₂. PAE-Flt1 cells (27) were from L. Claesson-Welsh (Uppsala University, Sweden) and maintained in Ham's F12 medium, 10% FBS, 5% CO₂. Primary HUVEC were freshly isolated as described (28) and cultured in Endothelial cell growth medium (PromoCell). Only cells passaged less than 3 times were used in this study. Most experiments were performed both with subconfluent and confluent HUVEC. HUVEC were transfected with DNA plasmids or siRNA duplexes by electroporation using Bio-Rad Gene Pulser Xcell (square-wave pulse) in Electroporation buffer (Ambion).

Immunofluorescence stainings. HUVEC were plated on type IV collagen, fixed with 4% paraformaldehyde and VEGFR2, TCPTP and α1 integrin were visualized using indirect immunofluorescence stainings. The samples were mounted in Vectashield with DAPI (Vector Laboratories, CA) and analyzed using confocal microscope (Axioplan 2 with LSM 510; Carl Zeiss MicroImaging, Inc.).

Immunoprecipitations. The plasmid for substrate-trapping mutant of TCPTP was cotransfected with a plasmid encoding VEGFR2 into HEK293 cells or transfected into PAE-Flt1 cells using Lipofectamine 2000. After 1 d the cells were serum-starved (overnight), stimulated with VEGF (50 ng/ml) for 10 min or left untreated, washed with ice-cold PBS and lysed. Postcentrifugation lysates were precleared with Protein-G-sepharose beads and subjected to immunoprecipitation with anti-TCPTP or control mouse IgG. After washings, the immunocomplexes were resolved on SDS-PAGE and subjected to immunoblotting. Aliquots of the original lysates (5% from IP input) were analyzed in parallel to control for protein expression.

Phosphatase and kinase assays. Phosphatase assays were performed as described (8). Briefly, HUVEC were plated to collagen or gelatin coated plates and allowed to adhere for 1 h. TCPTP, SHP2 and control immunocomplexes were isolated from cell lysates, resuspended in the phosphatase assay buffer and assayed for phosphatase activity using 6,8-difluoro-4-methylumbelliferyl phosphate; Molecular Probes) as a substrate in the presence of a serine/threonine phosphatase inhibitor cocktail (Sigma). Thereafter, the antigens from the beads were used for immunoblotting. The phosphatase assays with purified TCPTP protein were performed as described (8) using diFMUP as a substrate.

VEGFR2 kinase activity was studied using Kinase-Glo Plus kinase assay kit according to manufacturer's protocol (Promega) with 25 M ATP. Briefly, HUVEC plated on gelatin were serum-starved overnight, treated with 400 nM α1TAT or ScrTAT or left untreated and stimulated with 100 ng/ml VEGF for 15 min, when indicated. The cells were lysed to ice-cold lysis buffer (1% NP-40, 20 mM Hepes pH 7.4, 150 mM NaCl, 10% glycerol, 2.5 mM EDTA, complete protease inhibitor (Roche) and phosphatase inhibitor coctails I and II (Sigma)). The lysates were incubated in anti-VEGFR2 or control IgG-coated wells with shaking for 2 h and washed 3 times with lysis buffer and 3 times with cold kinase assay buffer (20 mM Hepes pH 8, 0.1 mM Na₃VO₄, 10 mM MnCl₂, 1 mM DTT). Kinase assay was performed for 10 min at +37° C.

For the in vitro dephosphorylation experiments, HEK293 transfected with VEGFR2 were treated with VEGF as described above. VEGFR2 was immunoprecipitated from the lysates (50 mM HEPES pH 7.5, 150 mM NaCl, 10% glycerol, 2.5 mM EDTA, 1% NP-40, Complete protease inhibitor cocktail, Sigma PPASE inhibitor coctails I and II). The beads were washed with the phosphatase assay buffer (10 mM Hepes, 150 mM NaCl, 1 mM DTT), and exposed to recombinant TCPTP (0.12 mg/ml) or buffer only for 10 min at +37° C. Immunoblotting was used to detect total VEGFR2 and specific phosphotyrosines.

Cell proliferation and survival assays. The number of viable HUVEC was analyzed with WST-1 reagent (Roche). Cell proliferation was measured with BrdU assay (Cell proliferation ELISA Biotrak system, Amersham) from siRNA-transfected HUVEC grown in RPMI1640+1% FBS+2 mM L-glutamine supplemented with 80 ng/ml VEGF.

Tube formation assays. In the fibrin-gel in vitro angiogenesis assay (Chemicon) HUVEC were plated onto a fibrin-gel, and overlaid with a second fibrin-gel after an overnight culture. In the collagen overlay assay (26) HUVEC were plated on gelatin-coated wells and allowed to attach. Then a collagen gel (0.2 M Hepes, 10×DMEM and Vitrogen 100 purified collagen I) was added. In both assays, after the polymerization of the top gel the endothelial cell growth medium with or without VEGF (50 ng/ml) was added in the presence or absence of α1TAT or scrambled TAT peptides (400 nM). At the 24 h end-point the formation of cellular networks was quantified from three randomly-selected fields from each well. In the fibrin gel assay scores 0-5 were assigned to each field based on cellular aligning, capillary tube formation, sprouting, and formation of polygons according to the manufacturer's instructions. In the collagen overlay assay the number of closed polygons formed by capillary tubes was quantified.

VEGFR2 internalization assay. HUVEC were transfected with control plasmid or with constitutively active 37 kDa TCPTP, grown on gelatin and stimulated with VEGF or left untreated. VEGFR2 internalization was followed microscopically (see above). For biochemical analyses cells were placed on ice and treated with cold trypsin (1 mg/ml, 30 min), which cleaves cell-surface expressed VEGFR2, but leaves internalized receptor intact (12). The amount of VEGFR2 in each sample was analyzed using immunoblotting.

Time-lapse chemokinesis assays. HUVEC were plated on gelatin-coated wells and allowed to attach for 1 h in the presence or absence of 200 nM α1TAT or ScrTAT and 20 ng/ml VEGF. Migration of individual cells was recorded at 5 min intervals for 4 h using 20× objective and digital video recording. The migration pathway of at least ten randomly picked cells was tracked using the Metamorph program. The cumulative migration distances were also plotted.

Chemotaxis assays. HUVEC electroporated with the DNA or siRNA or non-transfected HUVEC were treated with 200 nM α1TAT, ScrTAT or left untreated for 1 h. Transwell filters (8 μm pore size, Costar, Cambridge, Mass.) were coated with gelatin and medium (RPMI, 4 mM L-glutamine, 10% FBS) and VEGF (100 ng/ml) were added to the lower chamber. Cells were harvested from the culture plates, and transferred into the Transwell inserts (4×10⁵ cells/well) in RPMI with 0.2% soybean trypsin inhibitor. Chemotaxis was allowed to proceed for 4 h (+37° C., 5% CO₂). The inserts were removed, and the non-migrated cells from the top of the insert were removed by scraping with a cotton swab. The inserts were fixed in 4% paraformaldehyde, cells stained with 0.5% crystal violet and the number of migrated cells was counted using a microscope.

Statistical analyses. Statistical analyses were performed using Student's t-test.

### Results

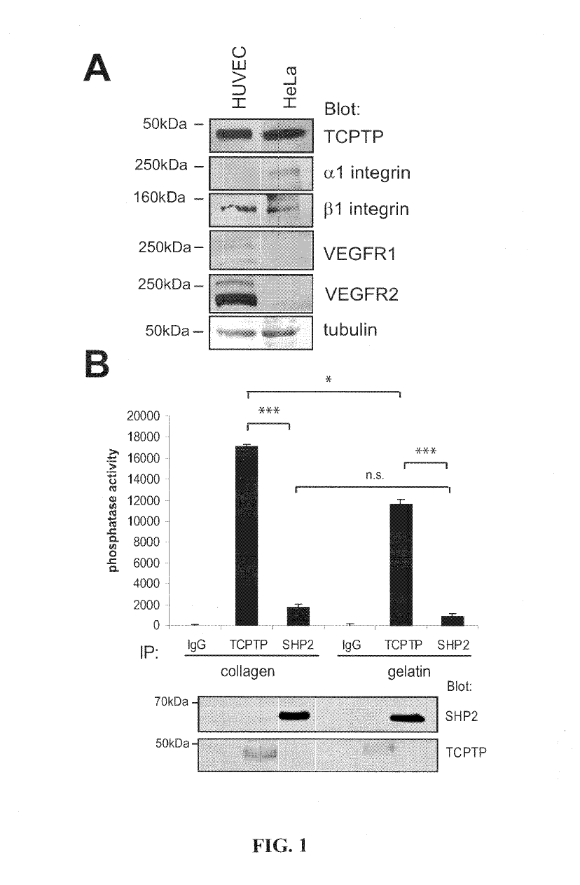

Adhesion of endothelial cells to collagen activates TCPTP. We hypothesized that human endothelial cells could express TCPTP and that adhesion to collagen via α1 integrin might affect its activity. Immunoblotting experiments revealed that TCPTP protein and α1β1 integrin are present in early-passage human umbilical vein endothelial cells (HUVEC; FIG. 1A). FACS-analyses further revealed that α1 integrin is expressed on the surface of HUVEC (not shown).

We then measured the phosphatase activity of TCPTP from HUVEC adhering to collagen (via α1 and α2 integrins) or to gelatin (mainly via αvβ3 integrin).

Immunoprecipitated TCPTP from HUVEC grown on gelatin showed high phosphatase activity when compared to a ubiquitous phosphatase SHP2. Notably, adhesion of HUVEC to collagen promoted activation of TCPTP by 47±1% (p<0.05), but it did not significantly increase SHP2 activity (FIG. 1B). Thus, HUVEC express α1β1 integrin and TCPTP and their binding to collagen matrix induces high TCPTP activity.

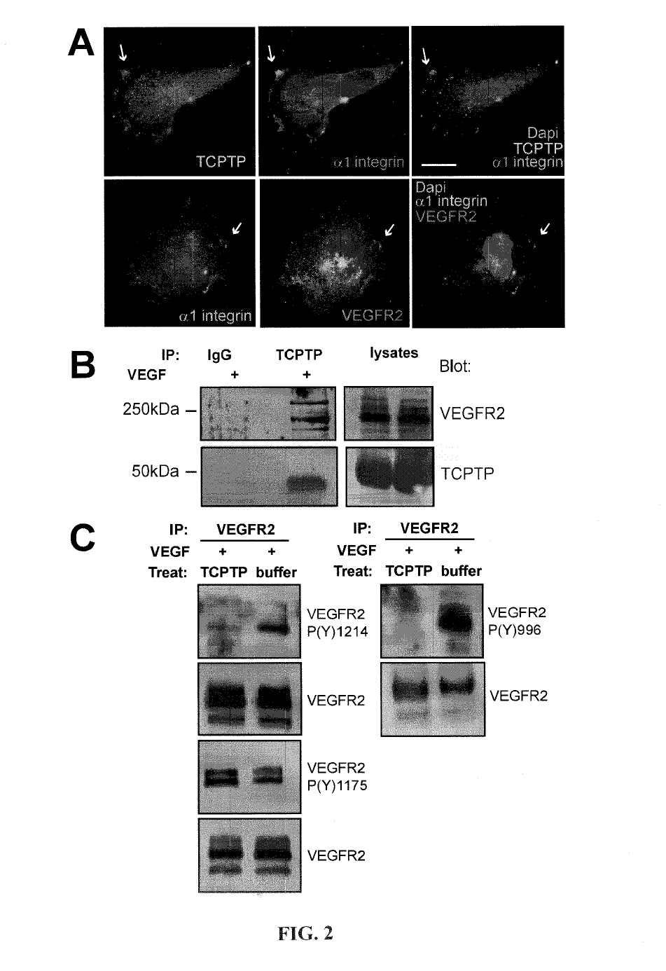

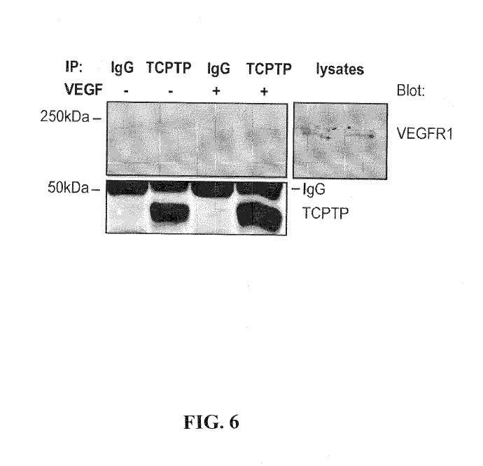

TCPTP binds VEGFR2 but not VEGFR1. To study whether α1 integrin, TCPTP and VEGFR could function as a signalosome we first determined their localization in endothelial cells. Immunofluorescence analyses showed that α1β1 colocalizes with TCPTP in HUVEC adhering to collagen (FIG. 2A). Interestingly, VEGFR2 also showed partial colocalization with α1 integrin in these cells (FIG. 2A). To test the binding of TCPTP to VEGFR we used a substrate-trapping technique. Binding of a substrate to TCPTP-D182A mutant results in a stable interaction between the enzyme and the substrate (9). We cotransfected the substrate-trapping mutant and VEGFR2 into cells and added human recombinant VEGF165 to induce receptor phosphorylation. VEGFR2 protein was readily co-immunoprecipitated with TCPTP-D182A (FIG. 2B). Interestingly, TCPTP-D182A did not associate with VEGFR1 in VEGF-treated cells overexpressing the receptor (FIG. 6). Thus, VEGFR2, but not VEGFR1, is a substrate for TCPTP in vivo.

TCPTP dephosphorylates VEGFR2 in a site-specific manner. VEGFR2 contains several critical tyrosine residues that are autophosphorylated or bind distinct effector molecules. Some of the most important ones are Tyr996 which is one of the autophosphorylation sites, Tyr1175 which binds to PLCγ and Shb, and Tyr 1214 which triggers p38 cascade through unknown intermediates (3). Phosphorylation assays showed that purified, recombinant TCPTP was able to dephosphorylate isolated VEGFR2 in vitro (FIG. 2C). Interestingly, TCPTP showed a striking specificity to two tyrosine residues studied (Y1214 and Y996), while tyrosine 1175 was not a TCPTP target. Thus, TCPTP binds to VEGFR2 both in vivo and in vitro and it dephosphorylates the receptor in a phosphosite-specific manner.

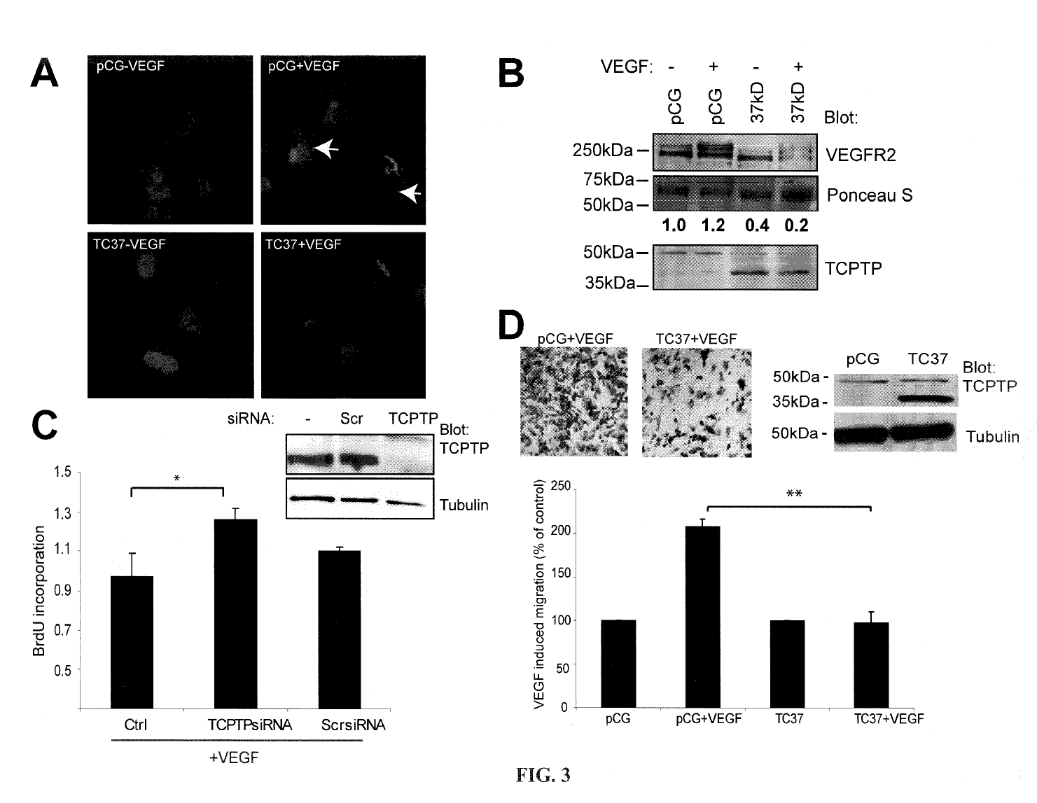

TCPTP controls internalization of VEGFR2. VEGFR2 is internalized upon engagement with VEGF and recent data suggests that internalized receptor is actively signalling (10,11). This prompted us to investigate whether TCPTP activity controls the internalization. VEGF treatment resulted in efficient endocytosis of VEGFR2 to early endosome-resembling vesicles in sparse mock-transfected HUVEC cells (FIG. 3A). Transfection of constitutively active 37 kDa TCPTP into HUVEC clearly inhibited VEGFR2 internalization to endosomes.

Cold trypsin treatment of HUVEC, which discriminates between the receptors exposed on the cell membrane (not protected from the cleavage) and the internalized receptors (protected from the cleavage) (12) was then used to quantify internalization biochemically. In sparse mock-transfected HUVEC, VEGF-stimulation efficiently increased VEGFR2 internalization, which was evident from the amount of trypsin-protected VEGFR2 detected by western blotting (FIG. 3B). In cells expressing the constitutively active TCPTP VEGF-induced internalization of VEGFR2 was almost completely blocked. These data thus show that in subconfluent cells activation of TCPTP leading to dephosphorylation of VEGFR2 results in the retention of VEGFR2 on the endothelial cell surface.

TCPTP activity regulates responsiveness of endothelial cells to VEGF. To study the biological effects of TCPTP activity on VEGFR2-mediated responses, we silenced TCPTP in endothelial cells using siRNA. In these cells VEGF-induced proliferation of HUVEC was 28±6% higher than in controls (FIG. 3C), which is consistent with the ability of TCPTP to dephosphorylated VEGFR2 in the absence of TCPTP. A constitutively active TCPTP was then used in chemotaxis assays to analyze the effect of enhanced VEGFR2 dephosphorylation on VEGF-driven cell migration. We found that VEGF stimulated chemotaxis of vector-transfected HUVEC by >2-fold. Strikingly, expression of the constitutively active TCPTP rendered the cells completely unable to migrate towards VEGF (FIG. 3D). Thus, decreased TCPTP activity enhances and increased TCPTP activity inhibits VEGF-driven responses in HUVEC.

The cytoplasmic domain of α1 integrin activates TCPTP and inhibits VEGFR-2 activity. We have demonstrated that in malignant epithelial cells a cell-permeable TAT-fusion peptide from the cytoplasmic tail of integrin α1, but not from that of α2, α5, α10 or α11 integrins, specifically activates TCPTP (8). Therefore, we studied whether this peptide could be used as a tool to trigger TCPTP-dependent VEGFR2 dephosphorylation in normal endothelial cells.

We observed that the FITC-labelled α1TAT fusion peptide and scrambled control peptide (ScrTAT) entered HUVEC within 30 minutes (data not shown). Subconfluent HUVEC that were treated with the scrambled control peptide or no peptide showed a prominent autophosphorylation of VEGFR2 at tyrosine 1214 upon VEGF-induction (FIG. 4A). This phosphorylation was markedly inhibited in HUVEC treated with the α1TAT peptide. We also noted that the scrambled TAT peptide induced a clear phosphorylation of VEGFR2, which is in line with the finding that TAT peptides bind VEGFR2 with a low affinity (13). Importantly, α1TAT peptide effectively attenuated this TAT-mediated induction of VEGFR2 phosphorylation as well (FIG. 4A). To test the functional significance of VEGFR2 dephosphorylation induced by α1TAT peptide, we measured the VEGFR2 kinase activities from HUVEC. Upon VEGF-stimulation the kinase activity increased about 10-fold in cells treated with no peptide or with the scrambled control TAT peptide. Strikingly, α1TAT peptide completely abolished the kinase activity of VEGFR2 in VEGF-treated HUVEC (FIG. 4B). Thus, the α1TAT peptide can be used to dephosphorylate and inactivate VEGFR2 in HUVEC.

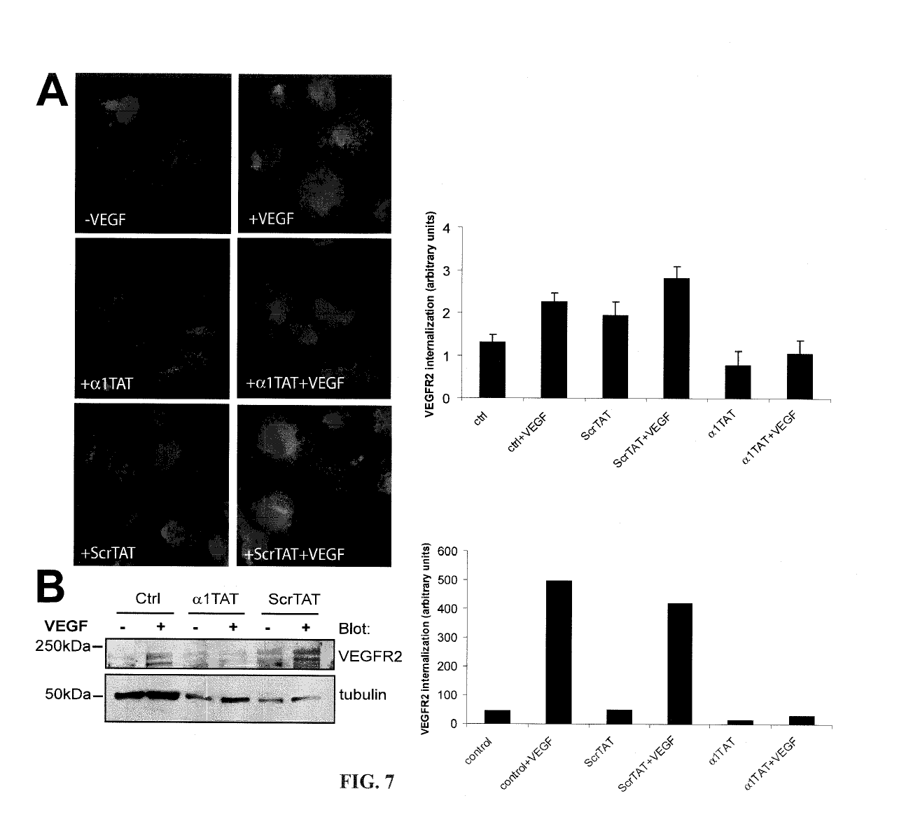

To increase the half-life of TAT-fusion peptides in cells, we designed novel TAT peptides with D-amino acids instead of normal L-isomers. In the in vitro phosphatase assay, α1-D-TAT was found to be a very good activator of TCPTP. Again the specificity of the TCPTP activation was evident, inasmuch as the scrambled TAT-fusion made of D-isomers had no effect (FIG. 4C). In a cell-based assay the α1-D-TAT specifically inhibited internalization of VEGFR2 in HUVEC (FIG. 7), which was consistent with the effects seen with constitutively active TCPTP.

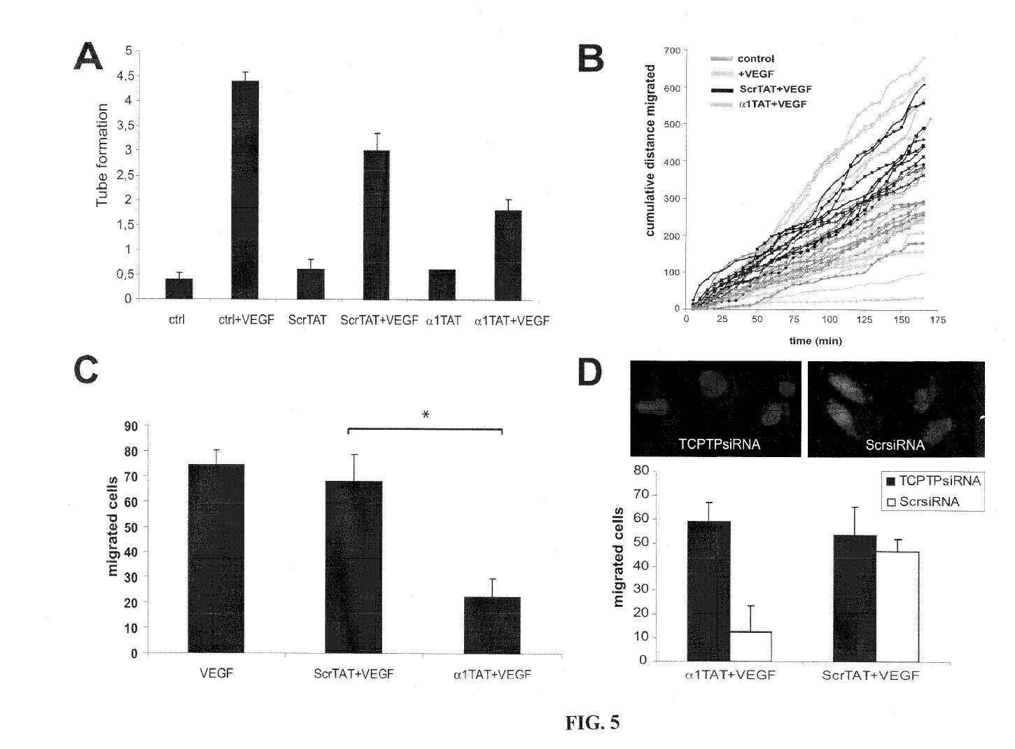

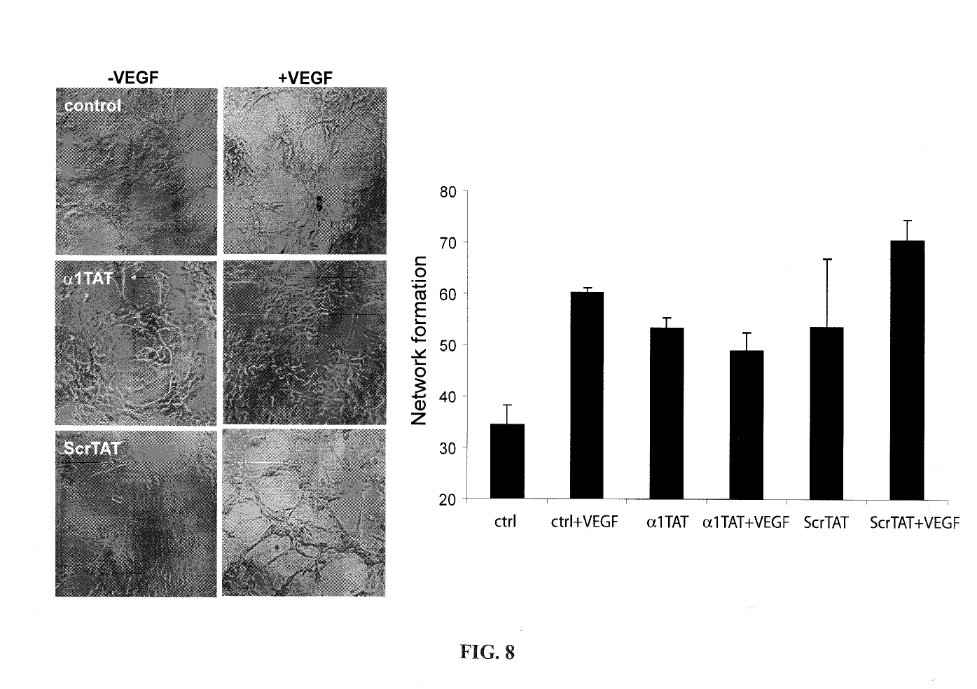

Controlled TCPTP activation can be exploited to inhibit endothelial proliferation, migration and capillary morphogenesis. Capillary morphogenesis requires adhesion, migration, proliferation, and differentiation of endothelial cells and VEGFR2 plays a major role in all of these steps (2). α1TAT peptide inhibited the capillary tube formation by 60% when compared to non-treated cells and by 40% when compared to control peptide treated cells in a fibrin gel assay (FIG. 5A). Similar inhibition of capillary formation by α1TAT peptide was found in a collagen overlay assay (FIG. 8). Together these results show that activation of TCPTP through the α1 cytoplasmic domain leads to inhibition of VEGF-driven capillary formation.

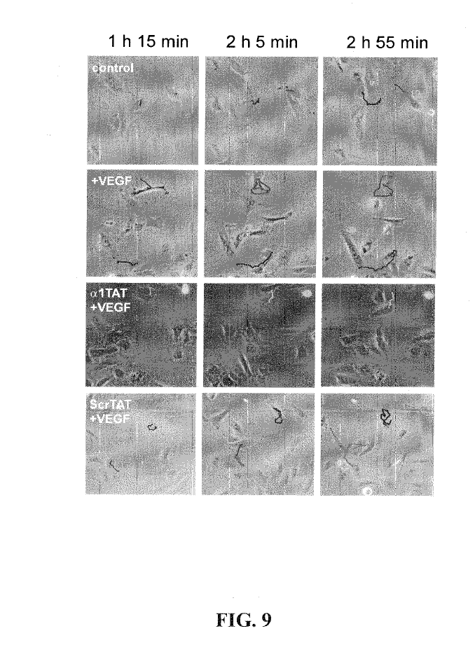

We next determined proliferation, chemokinesis and chemotaxis of HUVEC in response to the peptide treatment. The results showed that VEGF-induced proliferation of subconfluent HUVEC plated on gelatine was inhibited by α1TAT (74±8%; n=3; p<0.05 when compared to no peptide treatment), but not ScrTAT (4±4%; n=3; p>0.05). When the motility of HUVEC on gelatin-substrate was measured for 4 hours using time-lapse imaging, VEGF induced HUVEC chemokinesis by 99±25% (FIG. 5B and FIG. 9). In these assays, α1TAT treatment inhibited VEGF-induced chemokinesis by 76±4%, while ScrTAT had no significant effect. It was also clear from the images that neither TAT peptide was harmful to the cells, since all cells were viable and entered occasionally mitosis (not shown). VEGF-induced chemotaxis of HUVEC was also inhibited by treatment with α1TAT peptide, but not with the scrambled TAT peptide (FIG. 5C).

siRNA silencing was finally used to further confirm that the effect of α1TAT on VEGF-induced HUVEC migration is mediated via TCPTP. As shown in FIG. 5D, α1TAT had no inhibitory effects on the VEGF induced chemotaxis of TCPTP siRNA-treated cells, whereas α1TAT effectively inhibited VEGF-induced migration of scrambled siRNA treated cells. Together these data indicate that TCPTP is the target of the cytoplasmic peptide of α1 integrin in HUVEC and that decreased TCPTP activity results in augmented VEGFR2-mediated proliferation and migration of endothelial cells.

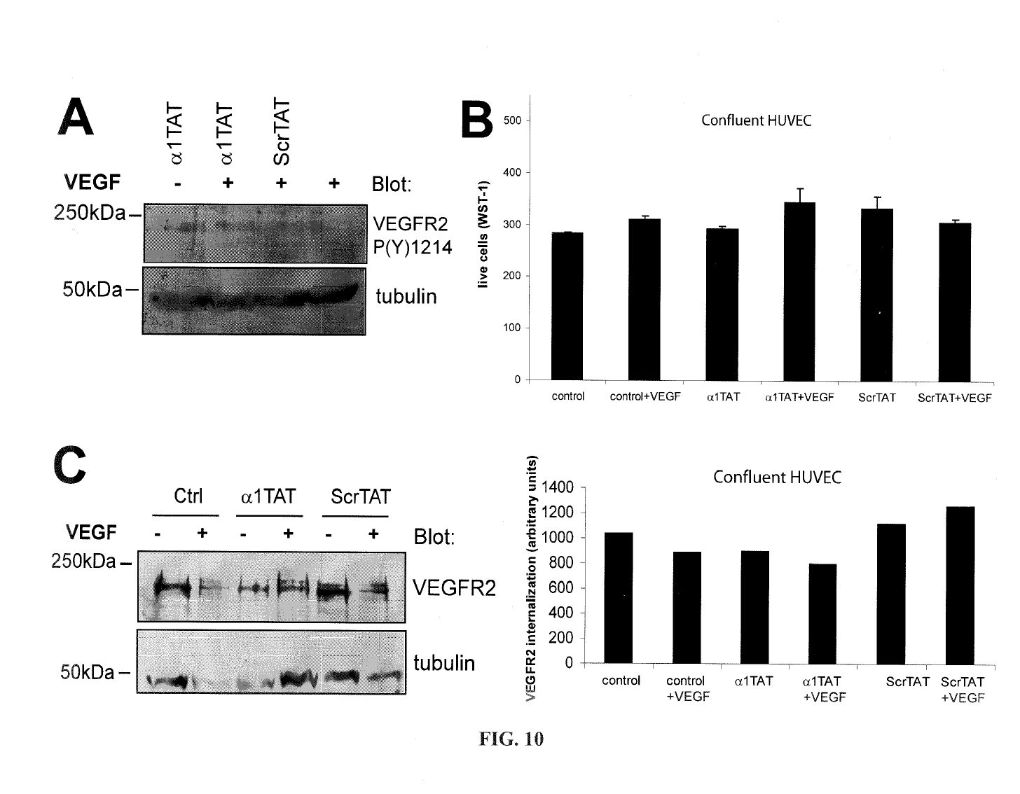

TCPTP activation selectively targets actively growing endothelial cells. An ideal anti-angiogenetic compound would spare normal confluent endothelial cells in mature vessels and only target newly forming vessels. In all previous experiments we used proliferating, subconfluent HUVEC. Therefore, we finally analyzed the effects of TCPTP on VEGFR2 signalling in confluent cells. These data showed that in confluent cells α1TAT and ScrTAT peptide-mediated TCPTP activation had no effects on VEGFR2 phosphorylation, survival of HUVEC, or VEGFR2 internalization (FIG. 10). Thus, in striking contrast to subconfluent cells, TCPTP activation has no effects in quiescent confluent endothelia.

### Discussion

The signalling pathways that down-regulate and terminate VEGF action are incompletely understood, although they are of paramount importance in controlling physiological and pathological angiogenesis. In this study we describe a novel phosphatase-mediated inhibition of endothelial cell proliferation, migration and capillary formation. We show that TCPTP binds to VEGFR2 and dephosphorylates its tyrosines in a phosphosite-specific manner. TCPTP activation diminishes the kinase activity of VEGFR2 and blocks its internalization from the cell surface. We also demonstrate that in endothelial cells the cytoplasmic domain of α1 integrin activates TCPTP. Moreover, a cell membrane-permeable peptide from α1-integrin efficiently attenuates VEGFR2-driven functions such as proliferation, migration, and tube formation in actively growing cells. Together these data show that TCPTP is a critical regulator of growth and differentiation of endothelial cells.

VEGF upregulates expression of α1β1 and α2β1 integrins in microvascular endothelial cells (14). In addition, a combination of function-blocking α1 and α2 antibodies inhibit VEGF-driven angiogenesis in mouse skin (14) and in tumor xenografts (15). We have shown that cross-linking of α1 integrin with the exactly same function-blocking monoclonal antibody leads to activation of TCPTP in malignant epithelial cells (8). Therefore, the inhibitory effects of anti-α1 antibodies on vascular growth in vivo may be due to antibody-triggered activation of TCPTP in addition to blocking α1β1 interaction with collagen.

Anti-VEGF antibodies are in clinical use and VEGFR2 kinase inhibitors are being developed for blocking neovascularization in several diseases (2). Our data suggest that inhibition of VEGFR2 activity by inducing TCPTP would be a novel strategy for antagonizing VEGF-activity. Since TCPTP-dependent VEGFR2 inactivation was only seen in subconfluent but not in confluent cells, its modulation might spare the normal vessels. Since TCPTP activation also directly inhibits the growth of epithelial cancer cells in vitro, it might simultaneously target both the malignant cells and the vessels nourishing them. To obtain suitable tools to test this hypothesis in vivo we have started to screen for novel small molecule TCPTP activators.

Upon VEGF stimulation, VEGFR2 is rapidly internalized and the rate of internalization is governed by phosphorylation (10,16). In contrary to the earlier dogma, internalized cell surface receptors maintain their signalling activity in the endosomes and even may recruit specific subset of down-stream effectors (17,18). In contact-inhibited cells, VEGFR2 is maintained inactive and prevented from being internalized by the VE-cadherin-β-catenin complex targeting VEGFR2 to the vicinity of density enhanced phosphatase-1 (DEP-1), which dephosphorylates the receptor and prevents its internalization (11). Once internalized, the receptor is actively signalling and phosphorylated at least on tyrosines 1175 and 1214 (11). SHP2 has also shown to interact with VEGFR2 presumably via its SH2 domain, but there are contradictory reports about its ability to dephosphorylate the receptor (19,20). Nevertheless, SHP2 has been reported to enhance internalization of VEGFR2 (20). In contrast, we showed here that in subconfluent cells TCPTP activation dephosphorylates VEGFR2 and prevents its internalization. The internalization routes for VEGFR2 are poorly characterized, but both clathrin-dependent and clathrin-independent mechanisms may be involved. Caveolin-1 codistributes with VEGFR2 (21,22) and may be involved in compartmentalization and inactivation of the receptor in the plasma membrane (16). Interestingly, α1 integrin is within a subclass of integrins that triggers Ras-pathway signalling via caveolin-1 (23). Therefore the cytoplasmic tail of α1 integrin may effectively inhibit VEGFR2 internalization either by dephosphorylating VEGFR2 through induction of TCPTP or by spatial regulation of the α1β1-VEGFR2-caveolin complex at the cell surface.

ECM proteins have been shown to positively regulate VEGFR2 activity in few cases. For instance, ligation of αvβ3 integrin supports VEGF-induced biological responses in endothelial cells in vitro and αvβ3 associates with VEGFR2 in response to VEGF treatment (19). Here we defined the first molecular pathway that leads to matrix-guided inhibition of endothelial cell responsiveness to VEGF. This pathway is triggered by binding of endothelial cells to collagenous matrix through integrin α1, which leads to activation of TCPTP and inhibition of VEGFR2 signalling. Our findings together with recent reports on the role of two other phosphatases in controlling VEGFR2 allows us to propose a general model of sequential, phosphatase-regulated steps in angiogenesis. (i) In intact endothelium, cells are contact-inhibited and unable to respond to VEGF. This is regulated by VE-cadherin-β-catenin complex that targets VEGFR2 to cell-cell contacts and results in its dephosphorylation by DEP-1 and possibly by other junctional phosphatases. In these confluent cells, ligation of α1β1 to basement membrane collagen IV and subsequent TCPTP activation would not have any additional effect since VEGFR2 is already functionally inactivated through DEP-1. (ii) Upon induction of angiogenesis, the sprouting endothelial cells will become surrounded by stroma ECM and ligated most likely via integrins αvβ3, α5β1, and α2β1 (which bind with high affinity to vitronectin, fibronectin and type 1 collagen, respectively). The cells are sensitive to VEGF, and VEGFR2 is fully phosphorylated and actively signalling to drive vessel sprouting. (iii) Upon formation of a new basement membrane, α1β1 integrin (binding to collagens IV and I) will activate TCPTP, and possibly SHP2, which begin to attenuate proliferation and migration of the endothelial cells until they reach confluency and become contact-inhibited (i). Based on this model, different phosphatases would inhibit VEGF signalling at different steps of angiogenesis. Pharmacological activator of TCPTP could thus be a safe and effective anti-angiogenic compound.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### REFERENCES

1. Coultas, L., Chawengsaksophak, K., and Rossant, J. (2005) Nature 438, 937-945
2. Ferrara, N., Gerber, H. P., and LeCouter, J. (2003) Nat Med 9, 669-676
3. Olsson, A. K., Dimberg, A., Kreuger, J., and Claesson-Welsh, L. (2006) Nat Rev Mol Cell Biol 7, 359-371
4. Giancotti, F. G. (1997) Curr Opin Cell Biol 9, 691-700
5. Moro, L., Venturino, M., Bozzo, C., Silengo, L., Altruda, F., Beguinot, L., Tarone, G., and Defilippi, P. (1998) Embo J 17, 6622-6632
6. Trusolino, L., Bertotti, A., and Comoglio, P. M. (2001) Cell 107, 643-654
7. Form, D. M., Pratt, B. M., and Madri, J. A. (1986) Lab Invest 55, 521-530
8. Mattila, E., Pellinen, T., Nevo, J., Vuoriluoto, K., Arjonen, A., and Ivaska, J. (2005) Nat Cell Biol 7, 78-85
9. Tiganis, T., Bennett, A. M., Ravichandran, K. S., and Tonks, N. K. (1998) Mol Cell Biol 18, 1622-1634
10. Dougher, M., and Terman, B. I. (1999) Oncogene 18, 1619-1627
11. Lampugnani, M. G., Orsenigo, F., Gagliani, M. C., Tacchetti, C., and Dejana, E. (2006) J Cell Biol 174, 593-604
12. Ceresa, B. P., Kao, A. W., Santeler, S. R., and Pessin, J. E. (1998) Mol Cell Biol 18, 3862-3870
13. Rubio Demirovic, A., Canadi, J., Weiglhofer, W., Scheidegger, P., Jaussi, R., and Kurt, B. H. (2003) Biol Chem 384, 1435-1441
14. Senger, D. R., Claffey, K. P., Benes, J. E., Perruzzi, C. A., Sergiou, A. P., and Detmar, M. (1997) Proc Natl Acad Sci USA 94, 13612-13617
15. Senger, D. R., Perruzzi, C. A., Streit, M., Koteliansky, V. E., de Fougerolles, A. R., and Detmar, M. (2002) Am J Pathol 160, 195-204
16. Labrecque, L., Royal, I., Surprenant, D. S., Patterson, C., Gingras, D., and Beliveau, R. (2003) Mol Biol Cell 14, 334-347
17. Di Fiore, P. P., and De Camilli, P. (2001) Cell 106, 1-4
18. Kermorgant, S., Zicha, D., and Parker, P. J. (2004) Embo J 23, 3721-3734
19. Kroll, J., and Waltenberger, J. (1997) J Biol Chem 272, 32521-32527
20. Mitola, S., Brenchio, B., Piccinini, M., Tertoolen, L., Zammataro, L., Breier, G., Rinaudo, M. T., den Hertog, J., Arese, M., and Bussolino, F. (2006) Circ Res 98, 45-54
21. Bhattacharya, R., Kang-Decker, N., Hughes, D. A., Mukherjee, P., Shah, V., McNiven, M. A., and Mukhopadhyay, D. (2005) Faseb J 19, 1692-1694
22. Ikeda, S., Ushio-Fukai, M., Zuo, L., Tojo, T., Dikalov, S., Patrushev, N. A., and Alexander, R. W. (2005) Circ Res 96, 467-475
23. Wary, K. K., Mainiero, F., Isakoff, S. J., Marcantonio, E. E., and Giancotti, F. G. (1996) Cell 87, 733-743
24. Tiganis, T., Kemp, B. E., and Tonks, N. K. (1999) J Biol Chem 274, 27768-27775
25. Matsumoto, T., Bohman, S., Dixelius, J., Berge, T., Dimberg, A., Magnusson, P., Wang, L., Wikner, C., Qi, J. H., Wernstedt, C., Wu, J., Bruheim, S., Mugishima, H., Mukhopadhyay, D., Spurkland, A., and Claesson-Welsh, L. (2005) Embo J 24, 2342-2353
26. Bix, G., Fu, J., Gonzalez, E. M., Macro, L., Barker, A., Campbell, S., Zutter, M. M., Santoro, S. A., Kim, J. K., Hook, M., Reed, C. C., and Iozzo, R. V. (2004) J Cell Biol 166, 97-109
27. Ito, N., Huang, K., and Claesson-Welsh, L. (2001) Cell Signal 13, 849-854
28. Koskinen, K., Vainio, P. J., Smith, D. J., Pihlavisto, M., Yla-Herttuala, S., Jalkanen, S., and Salmi, M. (2004) Blood 103, 3388-3395

1. Use of an agent capable of activating T cell protein tyrosine phosphatase (TCPTP) in the manufacture of a pharmaceutical composition for use in a method for inhibiting vascular endothelial growth factor receptor 2 (VEGFR2) signalling in an individual.

2. The use according to claim 1 wherein the agent is either i) a peptide comprising the amino acid sequence RPLKKKMEK (SEQ ID NO 1), or ii) a vector being capable of expressing said peptide in a mammalian cell, or iii) a small molecule able to activate TCPTP.

3. The use according to claim 2 wherein the agent is a peptide, which is administered as such or which is administered as a cell membrane permeable fusion protein.

4. The use according to claim 3 wherein the fusion protein comprises i) a protein transduction domain of HIV transcriptional transactivator (TAT) protein and the amino acid sequence RPLKKKMEK (SEQ ID NO 1), or ii) the antennapedia peptide RQIKIWFQNRRMKWKK (SEQ ID NO 6) and the amino acid sequence RPLKKKMEK (SEQ ID NO 1).

5. Use of an agent capable of activating T cell protein tyrosine phosphatase (TCPTP) in the manufacture of a pharmaceutical composition for use in a method for inhibiting angiogenesis or for treatment or prevention of a disease benefiting from suppression of the growth of blood vessels in an individual.

6. The use according to claim 5, wherein the disease is selected from the group consisting of retinopathy, age related macular degeneration, hemangioma, arthritis, psoriasis, atherosclerosis and cancer.

7. The use according to claim 6, wherein the disease is cancer and the tumor vasculature is destroyed or normalized.

8. The use according to claim 5, wherein the agent is either i) a peptide comprising the amino acid sequence RPLKKKMEK (SEQ ID NO 1), or ii) a vector being capable of expressing said peptide in a mammalian cell, or iii) a small molecule able to activate TCPTP.

9. The use according to claim 8, wherein the agent is a peptide, which is administered as such or which is administered as a cell membrane permeable fusion protein.

10. The use according to claim 9, wherein the fusion protein comprises i) a protein transduction domain of HIV transcriptional transactivator (TAT) protein and the amino acid sequence RPLKKKMEK (SEQ ID NO 1) or ii) the antennapedia peptide RQIKIWFQNRRMKWKK (SEQ ID NO 6) and the amino acid sequence RPLKKKMEK (SEQ ID NO 1).

11. The use according to claim 2, wherein all the amino acids in the peptide RPLKKKMEK (SEQ ID NO 1) are D-amino acids, and optionally all the amino acids in the fusion protein comprising the peptide RPLKKKMEK (SEQ ID NO 1) are D-amino acids.

12. Use of an agent capable of inhibiting T cell protein tyrosine phosphatase (TCPTP) in the manufacture of a pharmaceutical composition for use in a method for stimulating angiogenesis or for treatment or prevention of a disease benefiting from increasing the growth of blood vessels in an individual.

13. The use according to claim 12 wherein the agent is either i) small inhibitory RNA (siRNA) oligonucleotide capable of inhibiting TCPTP expression ii) a vector being capable of expressing said inhibitory RNA as a hairpin RNA (shRNA) in mammalian cell, or iii) a small molecule able of inhibiting TCPTP.

14. The use according to claim 12, wherein the disease is selected from the group consisting of coronary artery disease, peripheral arterial occlusion or another disease benefiting from promoting of neovascularisation.

15. A peptide comprising the amino acid sequence RPLKKKMEK (SEQ ID NO 1), wherein all the amino acids are D-amino acids, or a vector being capable of expressing said peptide in a mammalian cell.

16. The peptide according to claim 15, which is in the form of a cell membrane permeable fusion protein.

17. The fusion protein according to claim 16, which comprises i) a protein transduction domain of HIV transcriptional transactivator (TAT) protein and the amino acid sequence RPLKKKMEK (SEQ ID NO 1) or ii) the antennapedia peptide RQIKIWFQNRRMKWKK (SEQ ID NO 6) and the amino acid sequence RPLKKKMEK (SEQ ID NO 1).

18. The fusion protein according to claim 17, wherein all the amino acids in said fusion protein are D-amino acids.

19. The use according to claim 3, wherein all the amino acids in the peptide RPLKKKMEK (SEQ ID NO 1) are D-amino acids, and optionally all the amino acids in the fusion protein comprising the peptide RPLKKKMEK (SEQ ID NO 1) are D-amino acids.

20. The use according to claim 4, wherein all the amino acids in the peptide RPLKKKMEK (SEQ ID NO 1) are D-amino acids, and optionally all the amino acids in the fusion protein comprising the peptide RPLKKKMEK (SEQ ID NO 1) are D-amino acids.

21. The use according to claim 8, wherein all the amino acids in the peptide RPLKKKMEK (SEQ ID NO 1) are D-amino acids, and optionally all the amino acids in the fusion protein comprising the peptide RPLKKKMEK (SEQ ID NO 1) are D-amino acids.

22. The use according to claim 9, wherein all the amino acids in the peptide RPLKKKMEK (SEQ ID NO 1) are D-amino acids, and optionally all the amino acids in the fusion protein comprising the peptide RPLKKKMEK (SEQ ID NO 1) are D-amino acids.

23. The use according to claim 10, wherein all the amino acids in the peptide RPLKKKMEK (SEQ ID NO 1) are D-amino acids, and optionally all the amino acids in the fusion protein comprising the peptide RPLKKKMEK (SEQ ID NO 1) are D-amino acids.
